(19) United States
(12) Patent Application Publication
(10) Pub. No.: US 2001/0044652 A1
(43) Pub. Date: Nov. 22, 2001

(54) Stents with multi-layered struts

(76) Inventor: Brian Edward Moore, Los Altos Hills, CA (US)

Correspondence Address: Killworth, Gottman, Hagan & Schaeff, L.L.P., Suite 500, One Dayton Centre, Dayton, OH, 45402-2023, US

(21) Appl. No.: 09/882,466

(22) Filed: Jun. 14, 2001

Related U.S. Application Data
(63) Continuation-in-part of application No. PCT/US00/28385, filed on Oct. 13, 2000.
(60) Provisional application No. 60/159,319, filed on Oct. 14, 1999.

(51) Int. Cl. A61F 2/06

(52) U.S. Cl. 623/1.16; 623/1.17

(57) ABSTRACT

An endoluminal prosthetic device comprising axially repeating rings made up in turn of unit cells. The unit cells themselves are made up of circumferentially repeating patterns of multilayered strut members to form the ring. The rings may be axially connected to form a stent or an expandable housing for housing other medical device inserts. The multilayered struts, created by recessed slots cut from various regions in the strut members, permit improved radiopacity, increased flexibility during insertion stage into a lumen and better post-expansion conformability to the longitudinal shape of the body lumen, while providing increased rigidity and strain tolerance once the device has been expanded, as well as improved expansion ratio and fatigue characteristics. Variations on slot placement, length, orientation and shape with respect to the centerline of the strut members permit the optimization of a stent's strength, rigidity, strain and related mechanical properties. This approach reconciles competing needs for an expandable device to be low-profile and flexible enough to facilitate navigation through a tortuous body lumen so as to avoid causing lumen trauma prior to stent expansion and still achieve the expansion ratio and possess the needed radial strength to obtain and maintain lumen patency. Apertures placed in the struts further enable an easy and reliable point of attachment of an insert by means of sewing, stitching or riveting.

### CROSS REFERENCE TO RELATED APPLICATIONS

This application is a continuation in part of PCT Application Serial No. PCT/US00/28385, filed Oct. 14, 2000 (now International Publication Number WO 01/26584), which claims the benefit of U.S. Provisional Application 60/159,319, filed Oct. 14, 1999.

### BACKGROUND OF THE INVENTION

The present invention generally relates to an expandable endoluminal prosthetic device that can be used as a housing for attachment of a filter, drug release device, occlusion device, or valve in a vein or an artery, or as stent for an internal support function in an anatomical lumen, and more particularly to a device that exhibits improved flexibility in its unexpanded state combined with improved unit cell expansion and radial strength once expanded.

The class of medical devices that includes endoluminal prostheses, or stents, is generally known. For the purposes of this specification, the term “stent” shall encompass a broad meaning, referring to any expandable prosthetic device intended for implant in any body lumen. Therefore a stent can also be read as an expandable housing for attachment to a graft, filter, drug release device, urinary incontinence valve, occlusion device (such as a septal defect occluder or an intrafallopian contraceptive) for temporary or permanent use, and a valve in a vein or an artery, like a heart valve. In the present context, the most important function of the expandable device is a good anchoring in the anatomical lumen for prevention of leakage and axial migration. In general, stents are commonly used in the medical arts to internally support various anatomical lumens, such as a blood vessels, respiratory ducts, gastrointestinal ducts and the like. In addition, when expanded, their relatively rigid form can serve as a housing for other intraluminal devices, such as occlusion devices, filters, drug release devices, grafts and valves. Conventionally, stents are deployed in regions of stenosis or constriction in the target body lumen, and upon placement can be dilated by extrinsic or intrinsic means to hold the lumen open, thus obtaining a patent lumen and preventing immediate or future occlusion or collapse of the lumen and the resultant obstruction of fluids flowing therethrough. Because stent implantation is a relatively non-invasive procedure, it has proven to be a favorable alternative to surgery in many cases, for example, in certain cases of vascular stenosis.

Stents are typically made of biocompatible materials, and are comprised of numerous repeating geometric patterns, hereafter referred to as “unit cells”. Stents using unit cell pattern layouts have proven popular in the art, due in part to their mechanical simplicity and relative ease of manufacture. Such a configuration permits repeatable patterns to be incorporated into a thin layer of nonthrombogenic metal, metal alloy, durable plastic (such as polytetrafluoroethylene (PTFE), or biodegradable plastic (based on, among others, polyglycolic acid or polylactic acid)), or similar material, or combinations of any of these materials, arranged in a generally axisymmetric tubular shape. These patterns include a series of geometric shapes comprising strut members hingedly interconnected at axially and circumferentially periodic intervals. In the present context, “circumferential” can include helical patterns that traverse a path around a ring-like structure with both axial and purely circumferential components. Upon radial expansion of the stent, the strut members deform, being held together at these interconnection points, taking on a tubular/cylindrical cross section, thereby supporting the vessel walls from the inside.

Catheter-based delivery is the most common method of deploying a stent, while expansion of the stent is typically effected through one of two means, depending on the material properties and expansion characteristics of the stent to be implanted. For plastically deforming stents, such as those made from fully annealed 316L stainless steel, and certain elastic or superelastic stents, which are made from a biocompatible superelastic nickel titanium alloy, the expansion process is usually effected by placing the stent around a small expanding device, such as a balloon catheter, such that once the stent and catheter are inserted into the desired lumen location, the balloon can be inflated, forcing the stent to deform according to a predefined unit cell configuration. For self-expanding stents made from thermally-triggered shape memory materials or from elastic/superelastic materials, the stent is typically crimped over a delivery catheter and its closed shape is retained with a sheath. Once the catheter and stent have been properly located, the sheath is retracted and the stent expands to a predetermined expanded shape.

There are a few general performance characteristics that determine the overall functionality of a stent. First, in its unexpanded state, the stent must be flexible enough to allow navigation through tortuous anatomy to the target lesion. Second, it must be capable of an expansion ratio appropriate for the target anatomy, that is, it must be able to pass through the stenosis and it must radially expand to an appropriate size to achieve lumen patency. Additionally, it must be radially rigid enough to minimize the possibility of restenosis. Finally, it is desirable that a stent possess good radiopacity to facilitate visualization in the deployment, placement and expansion of the device.

One important measure of stent performance is expansion ratio, which is the diameter of the device after expansion compared to its diameter prior to expansion. The higher the expansion ratio, the more adaptable the stent is to use in anatomical lumens of varying size. Stent design has developed to a point where high expansion ratios can be achieved to yield devices with very small crossing profiles, which facilitates rapid and easy deployment, resulting in substantial advantages over early forms of the art. However, expansion ratios are limited by the level of strain introduced locally during the expansion process (whether in vivo or during manufacturing), often at or near the strut interconnection or hinge point. Conventional methods of increasing the expansion ratio of a stent to achieve a low-profile device while staying within acceptable localized strain limits include using longer and/or narrower expansion members, but these can result in diminished flexibility and/or decreased radial strength. Therefore, it would be desirable for a stent to achieve a greater expansion ratio for a given acceptable localized strain level without sacrificing flexibility or radial strength.

Another important performance characteristic for stents and related expandable housing is radial strength or rigidity. Different body lumens and different lesions may be such that a stent with extremely high radial strength is required to perform the task of obtaining and maintaining patency of the body lumen. Implanting a conventional stent without such characteristics may increase the potential for restenosis. Conventional stents may be modified to reduce the possibility of post-procedural narrowing or occlusion in the lumen by utilizing thicker and/or wider members to enhance the overall radial strength and rigidity. However, these bulkier members can not only impede delivery of the device by reducing its trackability, but are more prone to high localized strain levels, especially in the case where a plastically deformable stent is overexpanded to achieve a desired expansion ratio, which can lead to failure due to stress concentration, crack initiation and propagation, fatigue or accelerated corrosion. It is therefore desirable that a stent be able to achieve a greater radial strength or rigidity for a given acceptable level of localized strain, without compromising expansion ratio or longitudinal flexibility.

Still another desirable characteristic that may enhance overall stent usefulness is a useful level of radiopacity to facilitate visualization and placement of the device. Radiopacity may be enhanced by the use of a contrast medium, or by giving the stent structure a greater wall thickness. Unfortunately, application of a contrast medium complicates the manufacturing process. Additionally, use of a thicker-walled stent can increase the crossing profile of the device, thereby increasing the difficulty of deployment and navigation. Furthermore, since the cross-sectional aspect ratios of strut members can play an important role in longitudinal flexibility and stent trackability, altering these aspect ratios by increasing the wall thickness can lead to navigational and deployment difficulties by inhibiting the flexure of these members through tortuous anatomies. Therefore, a method of improving the radiopacity of a stent without the use of a contrast medium and/or without increasing its wall thickness is desired.

Accordingly, there is a need for a single expandable housing device that provides adequate structural properties, including strength, flexibility and expansion ratio at low localized strain levels, while simultaneously ensuring that procedures using such devices are simplified as much as possible.

### SUMMARY OF THE INVENTION

This need is met by the present invention wherein an expandable housing for inserting into an anatomical lumen comprises multiple strut layers that provide the added flexibility and inherently low strain levels of thin struts coupled with the radial strength and radiopacity of high cross-sectional aspect ratio struts. In general, the expandable housing for the attachment to a graft, filter, occlusion device, drug release device, urinary incontinence valve, occlusion device for temporary or permanent use, or a valve in a vein or an artery, like a heart valve, will be made as a single ring which is made of a series of circumferentially connected repeating open or closed unit cells. The present invention can also be made up of a plurality of axially interconnected rings, which, in turn are made up of circumferentially connected repeating unit cells. Moreover, the rings may be either closed (such that they do not connect axially), thereby functioning as a stand-alone structure, or open (such that they may interconnect axially) to form an axially elongate device. Variations in unit cell and ring structure would also permit a helical configuration. The unit cells themselves comprise a geometric pattern, and are made up of a plurality of interconnected, repeating strut members, which are in turn made up of hinge and lateral regions. One or more of the regions have recesses in or through their surfaces. Such recesses could be in the form of slots, ovals, circles, or some combination thereof In addition, the slots may be of continuous width, or may be tapered from one end to the other. By virtue of having multiple thin structures rather than a single thick structure made possible by the addition of the recesses, the expandable housing exhibits larger expansion ratios for a specified strain level and facilitates the growth of tissue around the strut members (or through the recesses in the strut members) as the tissue has less area to overcome. Moreover, the embodiments of the present invention avoid slot widening upon expansion of the unit cells. This is an important attribute, in that they act substantially as an anchor point, permitting the addition of or connection to other devices without ensuing interference upon unit cell expansion.

In accordance with a first embodiment of the present invention, a unit cell for an expandable housing is disclosed. The unit cell includes at least one hinge region and a plurality of lateral regions connected to the hinge region. The hinge region of the unit cell may be of either a plastically deformable configuration, or of a temperature-dependent shape memory alloy. The longitudinal dimension of the lateral regions can be slightly askew of the unit cell axial dimension. This can be valuable to minimize the amount of extra strain imposed on the unit cell when the unit cell is compressed to fit on a delivery device, such as a catheter. Moreover, the geometry of the lateral regions do not have to present a uniform shape; for example, the opposing lateral sides of the lateral regions do not have to be parallel to one another, such that a tapered configuration is possible, thus allowing for tailorable stress/strain behavior. The unit cell may optionally include a substantially elongate interconnect region with a proximal end that connects to either the hinge or lateral regions, and a distal end that can connect to a mating interconnect region in an adjacent unit cell. In the present context, the terms “elongate” and “substantially elongate” refer to a structural element that is markedly longer in its axial (lengthwise) dimension than in its sideways (widthwise) dimension. By having the interconnect region be of an elongate construction, separate from or in combination with locating it away from the hinge region, strain levels can be further reduced. The unit cell may also be fitted with a plurality of slots, which may further be discrete or continuous. In the present context, a “slot” is distinguished from an aperture in that it generally includes a large length-to-width ratio, whereas an aperture is either circular or mildly elliptical. Also in accordance with the present context, a slot is considered “discrete” when its lengthwise dimension does not traverse the entire length of the region in which it is disposed.

Neither the continuous nor the discrete slots, nor the struts in which they reside, need be of constant cross-section. For example, the slots are cut in such a pattern that the width of the adjacent strut members varies along their longitudinal direction, in order to have an optimized strain gradient over the strut length upon deformation by expansion. This can be achieved by several options. One possibility is cutting a symmetrical, longitudinally tapered slot with variable width in a strut with parallel outer edges, thus creating two identical tapered substruts at both sides of the slot. Another option is cutting an asymmetric longitudinally tapered slot in a strut with parallel outer edges. This can result in two adjacent substruts that have a different shape and taper, dependant on the expected strain levels upon expansion. An example is one prismatic substrut at one side of the slot and a tapered substrut on the other side, where in the present context, a “prismatic” member is one that has parallel opposing edges. Another possibility is to make a slot with parallel edges in a strut that was already tapered in longitudinal direction. A non-prismatic strut can be optimized to have a low stress concentration and/or low local strain on specific sections upon expansion. In such a configuration, the slot width is slightly variable upon expansion in order to keep the stress and strain levels in the adjacent substrut members lower than they would be in the case of rigid substrut connection. A slot with variable width can give way to a highly loaded hinge section, as well as allow some relative longitudinal movement between the adjacent substrut sections. This effect is well known in the construction of multi-layered leaf springs. This second order longitudinal movement between the layers gives a significant increase in the allowable amplitude of the spring in a direction perpendicular to the slots between the spring leafs.

Regarding the discrete slots particularly, the longitudinal axis (commonly known as the lengthwise dimension) of each of the discrete slots can be positioned asymmetrically with respect to the centerline of the region in which it is disposed. In such an asymmetrical configuration, the slot is either offset from the region's centerline, or is closer to one edge of the region than the other at a given lengthwise location of the region. The term “edge” refers to the outward-facing sides of the shortest (through-the-thickness) dimension of the region in question. Alternatively, the longitudinal axis of the disposed slots could be positioned equidistant from the edges of the region in which it is disposed, such that its orientation with respect to the region's centerline would be symmetric. Optionally, the plurality of slots could be positioned adjacent the lateral hinge points in the hinge region of one or more of the strut members.

Regarding the continuous slots particularly, they can alternatively be disposed within either the strut member's lateral or hinge regions. Furthermore, when disposed within the hinge region, the slot can have an exaggerated width in the vicinity of the hinge region's central hinge point. Moreover, the longitudinal axis each of the continuous, longitudinal slots can be positioned asymmetrically with respect to the centerline of the region in which it is disposed, or positioned equidistant from the edges of that same region. In addition, the plurality of slots could be positioned adjacent the lateral hinge points in the hinge region of one or more of the strut members. As with the discrete slots, the continuous slots can be tapered such that they define a variable spacing.

In accordance with another embodiment of the present invention, a generally tubular-shaped ring made up of circumferentially repeating unit cells for a stent is disclosed. The strut members of a unit cell making up each ring include at least one hinge region and a plurality of lateral regions, and optionally at least one interconnect region. The regions are made of generally thin, flat structural elements, and are either mechanically joined, or of a continuous construction. The strut member's regions may additionally include recesses similar to those of the previous embodiment. Furthermore, the ring may be either self-expanding (involving, for example superelastic materials or in a compressed spring-like state inside a restraining sheath) or non self-expanding (with separate inflation devices, such as a balloon catheter). In addition, the ring may be either of a plastically deformable configuration, or made from a temperature-dependent shape memory alloy similar to that of the previous embodiment. Upon the application of a radially outward-extending force on the tubular inner wall of the unit cell (in the case of non self-expanding configurations), or, upon removal of retaining sheath (in the case of self-expanding materials and configurations), from its lower diameter first state to a larger diameter second state, the circumferential dimension of the unit cell increases to an amount predetermined by the unit cell's expansion ratio.

In accordance with another embodiment of the present invention, a generally tubular-shaped ring including at least one hinge region, a plurality of lateral regions and at least one elongate interconnect region, where one or more recesses similar to those of the previous embodiment are disposed through the surface of at least one of the regions. In the present embodiment, the lateral regions are angularly offset from the axial dimension of the ring. The more compact arrangement made possible by avoiding a parallel construction between the ring's axial dimension helps to minimize the amount of additional strain placed on the ring when it undergoes compression to fit on or in a delivery device, such as a catheter. As with the previous embodiments, the ring may be either self-expanding or non self-expanding, and can additionally be of either a plastically deformable or shape memory alloy configuration. Also as with previous embodiments, the recesses can comprise various discrete or continuous slot configurations, and can be disposed in either symmetric or asymmetric ways.

In accordance with yet another embodiment of the present invention, a stent with a plurality of axially repeating rings is disclosed. As with the rings mentioned in the previous embodiment, the stent can be either self-expanding or non self-expanding, and can either be of a plastically deformable configuration or one that utilizes shape memory alloys. The stent comprises a plurality of axially interconnected rings, made up of circumferentially interconnected unit cells. The unit cells, which can be configurationally similar to those of any of the previous embodiments, can be axially connected to one another via the hinge or lateral regions, or at any location in between. In addition, axial connection can be effected by the optional interconnect regions, where the adjacent distal ends can be mated. In either case, adjacent unit cells can be either mechanically joined to, or made in continuous construction with, one another. Particular slot and lateral region configurations, as discussed in conjunction with the first embodiment, may also be incorporated. Moreover, the earlier discussed recesses can serve additional functions, such as the attachment of stitches, sewing wire or rivets that connect the stent structure to a graft material that is to be placed into the body lumen together with the stent. Accordingly, the geometry of the slot may be locally adapted to enable an easy and reliable attachment of such stitches, sewing wire or rivets without adversely effecting the stent expansion and crimping characteristics. This is an improvement over existing stents, where stitches are attached around the struts and interfere with each other when adjacent struts come closer to each other upon crimping of the stent. Further, the slots can be used for the attachment in the mentioned applications for housings of filters, drug release devices, occlusion device and valves.

In accordance with another embodiment of the present invention, a stent with a plurality of repeating unit cells, each with strut members defined by at least one hinge region, a plurality of angularly offset lateral regions, and at least one elongate interconnect region, with slot-shaped recesses disposed in at least one of these regions, is disclosed. As with one of the ring embodiments discussed earlier, the offset angle of the struts can reduce stresses placed on the various regions when the device is crimped to fit onto or inside a delivery device. The struts, unit cells and rings making up the present embodiment can be configurationally similar to those of any of the earlier embodiments, incorporating their salient features.

In accordance with another embodiment of the invention, an expandable medical device configured for use as a housing for intraluminal inserts is disclosed. The expandable medical device includes a first state defining a first diameter and a second state defining a second diameter, wherein the second diameter is greater than the first diameter by an amount defined by a predetermined expansion ratio. The expandable medical device comprises a plurality of continuous strut members arranged to define a generally repeating pattern, wherein the generally repeating pattern is arranged such that each of the continuous strut members comprise a plurality of regions, and at least one slot is disposed in at least the hinge region or the plurality of lateral regions. The plurality of regions includes a hinge region, a plurality of lateral regions each in connection with the hinge region, and an interconnect region to connect the generally repeating pattern with an adjacent repeating pattern. The expandable medical device of the present embodiment is especially well configured to provide an anchor for other intraluminal inserts, such as a valve, occlusion device, drug release device, filter or graft material. Optional features may include at least one aperture disposed in at least one of the plurality of regions to facilitate the attachment of the expandable medical device an insert selected from the group consisting of graft material, valves, occlusion devices, drug release devices and filters. These apertures are to be distinguished from the slots, in that while both result in a space within an otherwise solid member, the slots are primarily intended for stress/strain reduction upon expansion, and the apertures are used primarily to establish connection between the expandable device and an insert device designed to be anchored to the expansion device. Preferably, attachment to the inserts is effected through the use of conventional attachment schemes, including stitches, sewing wire and rivets.

In accordance with still another embodiment of the present invention, a method for expanding an expandable housing with a plurality of repeating rings is disclosed. The method comprises configuring a generally tubular expandable housing with a plurality of circumferentially interconnected unit cells comprising axially interconnected rings. The method of expansion may vary, depending on if the expandable housing is self-expanding or non self-expanding. In the case of a non self-expanding housing, a catheter is inserted inside the tubular inner wall of the housing. Fluid pressure is then applied to the catheter, which expands, applying radially outward-extending pressure to the inner wall of the tubular housing, which then expands a predetermined amount. Once the expansion is complete, the fluid pressure force on the catheter is removed, causing the catheter to deflate, at which time it can be withdrawn from the now expanded housing. In the alternative involving a self-expanding housing, the housing is typically crimped over a delivery catheter and its closed shape is retained with a sheath. Upon placement of the housing in its desired location, the sheath is retracted, allowing the housing to expand to a predetermined expanded shape. In either the self-expanding or non self-expanding variants, optional slots of the kind discussed in conjunction with the previous embodiments may be included in at least some of the strut regions. Also as before, the lateral region portions of the struts may be angularly offset relative to the axial dimension of the expandable housing.

In accordance with another embodiment of the invention, a bistable unit cell to be used in a stent for inserting into an anatomical lumen and expandable upon insertion into the lumen is disclosed. The unit cell includes a solid elongate strut member, a slotted elongate strut member capable of a first bistable state and a second bistable state, and a plurality of axially spaced hinge members. The slotted elongate strut member is nested with the solid elongate strut member in the first bistable state, and expanded away from the solid elongate strut member in the second bistable state. Each hinge is defined by joined adjacent ends of the solid elongate strut member and the slotted elongate strut member, while the plurality of axially spaced hinge members define an elongate aperture that is configured as a curvilinear slot in the first bistable state, and as an expanded opening in the second bistable state. The use of shape memory alloys, such as nickel-titanium, is especially beneficial in conjunction with the bistable configuration of the present invention, as the temperature dependency of the shape memory material can be engineered to cause the unit cell to expand from its first bistable state into its second bistable state at a controlled or prescribed temperature condition.

In accordance with still another embodiment of the invention, a bistable ring for use in a stent for inserting into an anatomical lumen and expandable upon insertion into the lumen is disclosed. The bistable ring includes a plurality of circumferentially repeating unit cells, each comprising a solid elongate strut member, a slotted elongate strut member capable of a first bistable state and a second bistable state, a plurality of axially spaced hinge members each defined by joined adjacent ends of the solid elongate strut member and slotted elongate strut member, and an interconnect region disposed between adjacent bistable unit cells such that the adjacent bistable unit cells are configured to cooperatively expand when the bistable ring expands from its first bistable state into its second bistable state. As with the previous embodiment, the plurality of axially spaced hinge members define an elongate aperture configured as a curvilinear slot in the first bistable state, and as an expanded opening in the second bistable state.

In accordance with yet another embodiment of the invention, a bistable stent cell for inserting into an anatomical lumen and expandable upon insertion into the lumen is disclosed. The bistable stent includes a plurality of axially aligned, circumferentially interconnected bistable rings made up of individual unit cells. Each of the unit cells include a solid elongate strut member, a slotted elongate strut member capable of a first bistable state and a second bistable state, a plurality of axially spaced hinge members, each defined by joined adjacent ends of the solid elongate strut member and slotted elongate strut member, a first interconnect region disposed between adjacent bistable unit cells, and a second interconnect region disposed between axially adjacent hinge members such that said plurality of axially aligned bistable rings are connected together to form said bistable stent. The plurality of axially spaced hinge members define an elongate aperture configured as a curvilinear slot in the first bistable state and as an expanded opening in the second bistable state. The slotted elongate strut member is nested with the solid elongate strut member in the first bistable state, and expanded away from the solid elongate strut member in said second bistable state. The first interconnect region is such that the adjacent bistable unit cells are configured to cooperatively expand when the bistable ring expands from the first bistable state into its second bistable state.

These and other objects of the present invention will be apparent from the following description, the accompanying drawings and the appended claims.

### BRIEF DESCRIPTION OF THE SEVERAL VIEWS OF THE DRAWINGS

The following detailed description of the preferred embodiments of the present invention can be best understood when read in conjunction with the following drawings, where like structure is indicated with like reference numerals and in which:

FIG. 1 is an isometric view of a stent according to an embodiment of the present invention in an unexpanded state;

FIG. 2 is an isometric view of the stent of FIG. 1 in an expanded state;

FIG. 3 is a top view of a portion of a unit cell of a stent according to an embodiment of the present invention, depicting discrete slots asymmetrically disposed in some of the strut members;

FIG. 4 is a top view of a portion of a unit cell of a stent according to an embodiment of the present invention, depicting discrete slots disposed equidistant between the edges of some of the strut members;

FIG. 5 is a top view of a portion of a unit cell of a stent according to another embodiment of the present invention, depicting continuous, longitudinal slots asymmetrically disposed in the hinge region of the strut members;

FIG. 6 is a top view of a portion of a unit cell of a stent according to another embodiment of the present invention, depicting continuous, longitudinal slots asymmetrically disposed in the lateral region of the strut members;

FIG. 7 is a top view of a portion of a unit cell of a stent according to another embodiment of the present invention, depicting continuous, longitudinal slots disposed equidistant between the edges of the lateral region of the strut members;

FIG. 8 is a top view of a portion of a unit cell of a stent according to another embodiment of the present invention, depicting continuous, longitudinal slots disposed equidistant between the edges of the hinge region of the strut members;

FIG. 9 is a variation of the unit cell of FIG. 8, where the slot is exaggerated near a central hinge in the hinge region;

FIG. 10A is an end view of a bistable unit cell of the stent in an expanded stable position according to an embodiment of the present invention;

FIG. 10B is an end view of the bistable unit cell of FIG. 10A in a collapsed stable position;

FIG. 10C is an isometric view of a single stent ring in a collapsed state, incorporating the features of the unit cell of FIG. 10A;

FIG. 10D is an isometric view of the single stent ring of FIG. 10C in an expanded state;

FIG. 11A shows a tapered slot that divides a prismatic lateral region into two similar substruts;

FIG. 11B shows a tapered slot that divides a prismatic lateral region into two dissimilar substruts;

FIG. 11C shows a prismatic slot that divides a tapered lateral region into two similar substruts;

FIG. 11D shows adjacent struts that can be configured as a leaf spring, in an unbent configuration;

FIG. 11E shows the adjacent struts of FIG. 11D under a bending condition when the two struts are not joined together;

FIG. 11F shows the adjacent struts of FIG. 11D under a bending condition when the two struts are joined together, producing a fold and gap;

FIGS. 12A-12C show an expandable housing configured to hold a valve; and

FIGS. 13A-13C show an expandable housing configured to hold a filter.

### DETAILED DESCRIPTION OF THE PREFERRED EMBODIMENTS

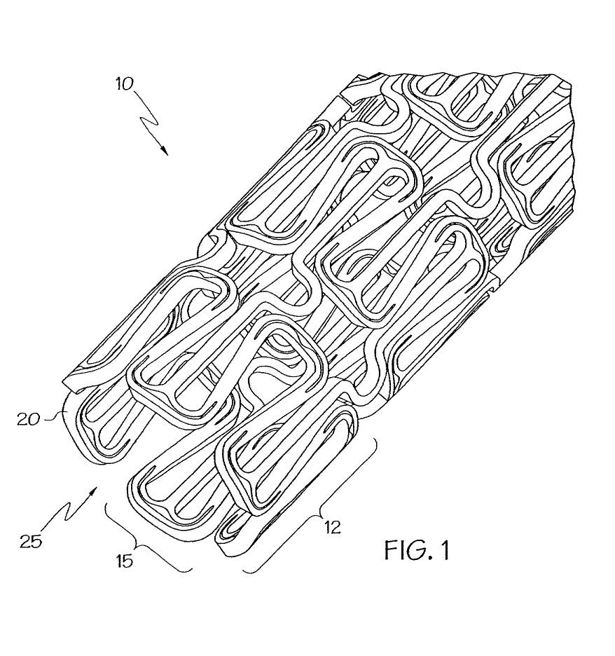

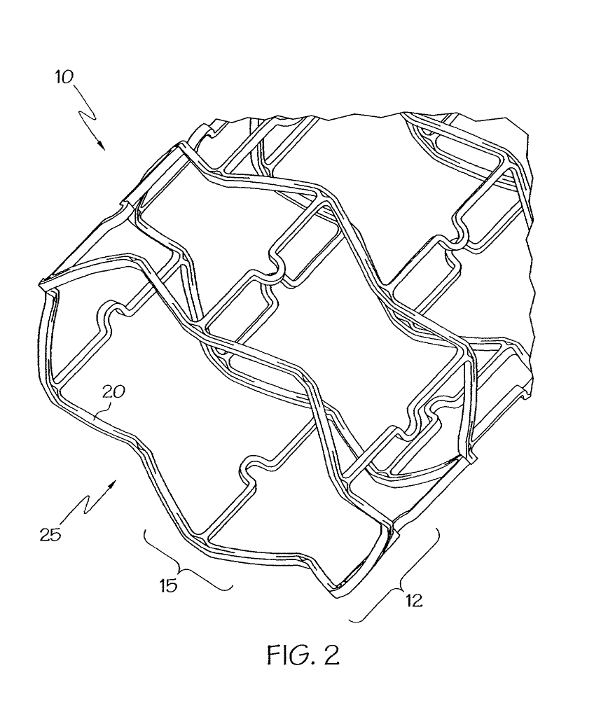

Referring now to FIGS. 1 and 2, an expandable housing 10 (alternately referred to as a stent) comprises a plurality of axially repeating rings 12, which are made up of circumferentially and continuously interconnected unit cells 15, which are in turn made up of strut members 20. The plurality of rings 12, unit cells 15 and strut members 20 define an exoskeletal main support structure of the stent 10. The stent 10 is of generally tubular construction, defined by a hollow internal portion 25. The strut members of the unit cell may either be from a continuous piece of material, or be connected by any conventional joining approach, such as hinging, welding, gluing, or the like. By extrapolation, the plurality of rings 12 and unit cells 15 making up stent 10 can also be of a single sheet of material, or a combination of individual pieces. FIG. 1 shows the stent in an unexpanded state. The construction of the unit cells 15 is such that as a radially outward-extending force is applied to the tubular internal portion 25, the stent's diameter D increases, resulting in an expanded state, as shown in FIG. 2. One conventional form of expanding force is a balloon catheter (not shown), which is first inserted axially into the hollow internal portion 25, followed by the application of hydraulic or pneumatic pressure from an external supply. Another form (not shown) of expanding force can come from the stent itself, in the form of a thermally-triggered shape memory material. Like the balloon catheter approach, it is first inserted into the desired lumen location. However, unlike the balloon approach, a retaining sheath is placed on the outside of the stent to keep it in its compressed state. Once the sheath is removed, the stent expands to its predetermined configuration.

The strut members 20 of stent 10 are the load-carrying elements in the unit cell 15; thus, upon the relatively uniform application of force from the balloon, localized deformation takes place at the various hinge points (discussed in more detail below) in the strut members 20. The unit cells 15 are chosen based on constitutive material properties in addition to desired as-expanded size, for example, if a stent is to be manufactured from a fully annealed 316L stainless steel tube, the unit cells are designed so as to ensure that the hinge points deform beyond their elastic limit to avoid the occurrence of stent recoil, which could otherwise cause the stent 10 to dislodge and migrate to a downstream portion in the lumen.

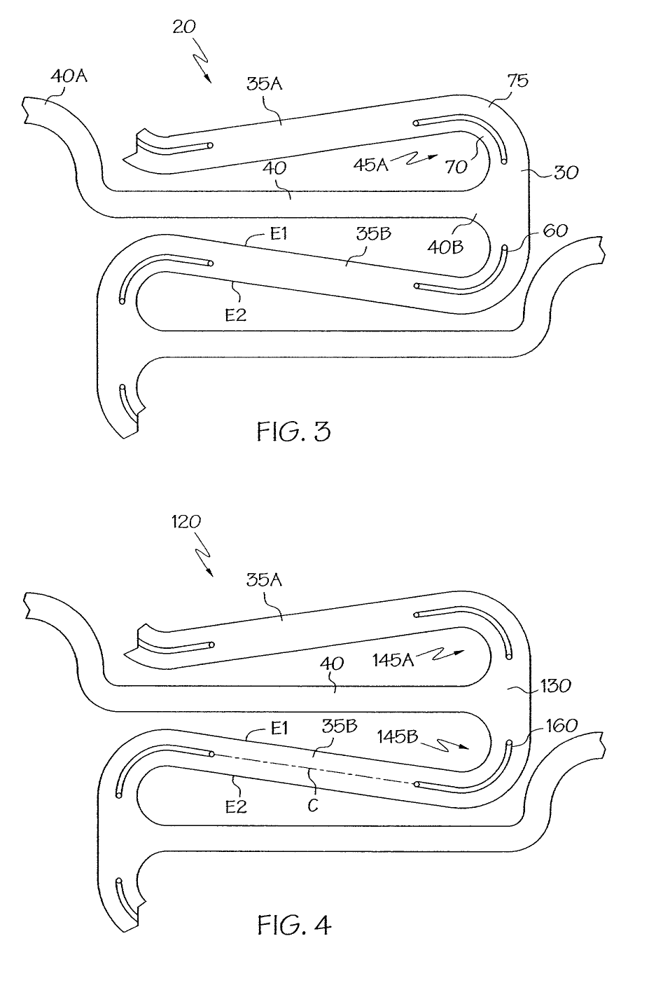

Referring now to FIG. 3, strut member 20 is made up of multiple regions, including a hinge region 30, one or more lateral regions 35A, 35B roughly aligned with the axial direction of the stent, and an interconnect region 40. The widthwise dimensions of all of the regions are bounded by opposing edges E1 and E2 (shown only on lateral region 35A, but representative of all regions) that span the entire length of each of the regions. Lateral regions 35A, 35B of each strut member maintain circumferential connection between adjacent unit cells, while the distal end 40A of interconnect region 40 maintains axial connection with other unit cells in axially adjacent rings (not shown). The ends of the lateral regions 35A, 35B meet corresponding ends in the hinge region 30 at lateral hinge points 45A and 45B, while the proximal end 40B of interconnect region 40 meets either substantially in the center of the hinge region 30 (as shown), or along one of the sides of the lateral regions 35A, 35B. Upon radial expansion of stent 10, the lateral regions bend away from the stent axis, causing lateral hinge points 45A and 45B and central hinge point 50 to act as a hinge. Full expansion of the unit cell 15 is designed to be accompanied by plastic deformation in the hinge region 30. To meliorate the localized strain caused in the hinge region 30 by the expansion process, recesses are cut into portions of the hinge region 30, resulting in “multilayered” strut members. Thus, in looking widthwise from one edge to the other through a region with a recess disposed therein, one would “see” two separate sections 70 and 75. Similarly, in this multilayered configuration, an applied force encounters two thinner structural members in series, rather than one thicker member. This has the advantage of providing virtually the same strength as the “one-piece” (or single-layered) member, but with dramatically greater strain tolerance. In the preferred embodiments of the present invention, the recesses are longitudinal cuts, or slots 60, inserted into the strut members 20, although it is recognized that other shapes, such as circles and prolate and oblate ellipsoids, could also be used. Preferably, the slots 60 would constitute elongate slots that penetrate the entire thickness of strut 20. While two individual layers are shown and described, it is within the scope of the present invention to use a greater or lesser number to achieve the desired structural response.

Asymmetric placement of the slot between the opposing edges E1 and E2 can be optimized to promote a balanced strain profile between sections 70 and 75. In addition to providing greater strain tolerance, the slots 60 help to achieve a level of flexibility necessary to ensure that the stent 10 can be inserted into a curved section of a lumen (not shown) without puncturing or otherwise damaging the lumen wall. While the material can typically be any biocompatible material, such as stainless steel, titanium, gold, nickel-titanium (often called shape-memory metal or “nitinol”) alloys, plastics and the like, the invention described herein could also consist of a hybrid material approach, wherein multiple metal alloys, or metal-plastic combinations, or even organic-, metal- or ceramic-matrix composites could be used.

Different embodiments of the above-mentioned approach will now be described. Turning now to FIG. 4, the main difference between this embodiment and that of FIG. 3 is with the placement of the discrete slots 160. In the present embodiment, the slots are placed along the centerline C such that the slot 160 is equidistant from opposing edges E1 and E2. Whereas the embodiment of FIG. 3 includes slots placed asymmetrically such that the slots are closer to one edge (in this case E2) than the other. Advantages associated with this approach include reduced manufacturing cost, as well as higher strength. It is also noted that with this embodiment, as well as the others where lengthy or numerous slots are incorporated, endothelial tissue growth could be promoted by adding additional apertures or slots along portions of strut member 20 that are not subject to deformation during the expansion process. Such slot schemes could also promote growth opportunities with other forms of tissue. These slots may also be helpful for the attachment of graft material, by sewing, stitching or riveting.

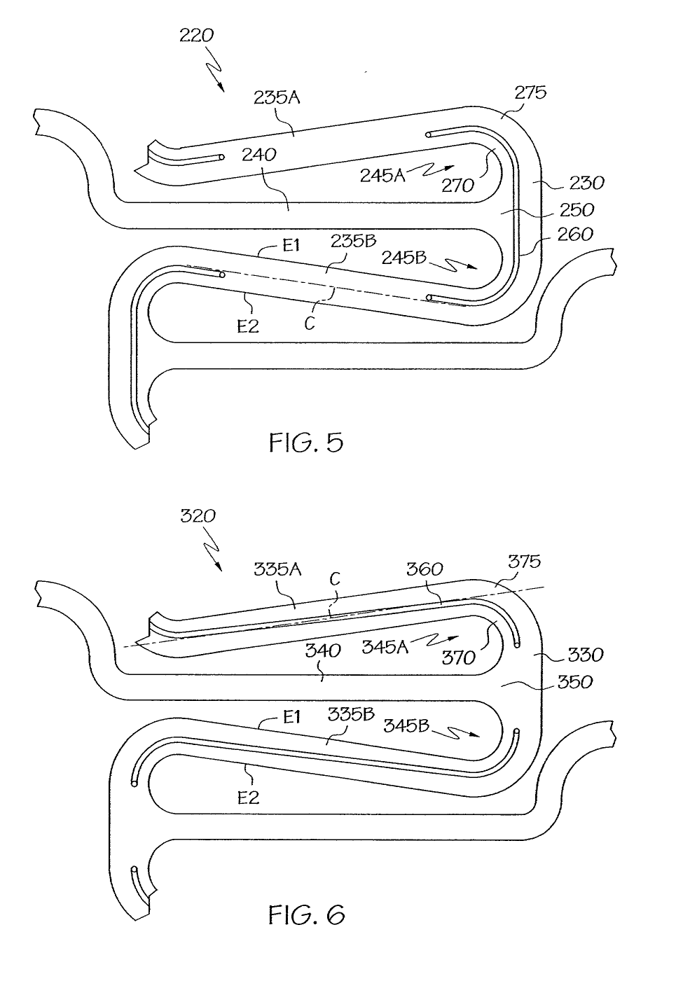

Referring now to FIG. 5, a continuous, longitudinal slot 260 is disposed in an offset relationship from centerline C, which in the present context is an imaginary line that traverses throughout the length of the region equidistant between the opposing edges E1 and E2. In the present context, a slot is considered “continuous” if it extends uninterrupted across the entire length of the region in which it is disposed, spanning over at least partially into an adjacent region. The continuous slot is to be contrasted with the “discrete” slot that has a pattern that, while still occupying both the region in which it is disposed and at least a part of adjacent regions, is discontinuous such that a solid bridge of material extends from edge-to-edge in at least widthwise part of the region. Accordingly, the instant configuration is different from that shown in FIG. 3 in that the slot extends uninterrupted all the way through the hinge region 230, including all of the strain-intensive hinge points 245A, 245B and 250. As with other asymmetrical features (such as that shown in FIG. 3), balanced strain profiles are possible. An advantage to having the multiple layers 270, 275 extend through the entirety of the hinge region 230 is that strain-relief features can be maximized, while still providing adequate strength characteristics in the strut members 220.

Referring now to FIG. 6, a continuous, longitudinal slot 360 is disposed in the lateral regions 335A and 335B. As with the embodiment shown in FIG. 5, the slot 360 is disposed in an skewed relationship with the axis of the centerline C, resulting in an asymmetrical positioning. Note in particular that this skewed positioning allows the slot to provide both continuous strain relief along the entire length of the lateral regions 335A and 335B, as well as maintaining a balanced strain profile by having more structure removed from the inner hinge points 370 than the outer 375. This allows the wider (and hence, stronger) outer section 375 to carry the majority of the tensile bending load caused when the expanded stent 320 is subjected to a compression load, such as from the lumen.

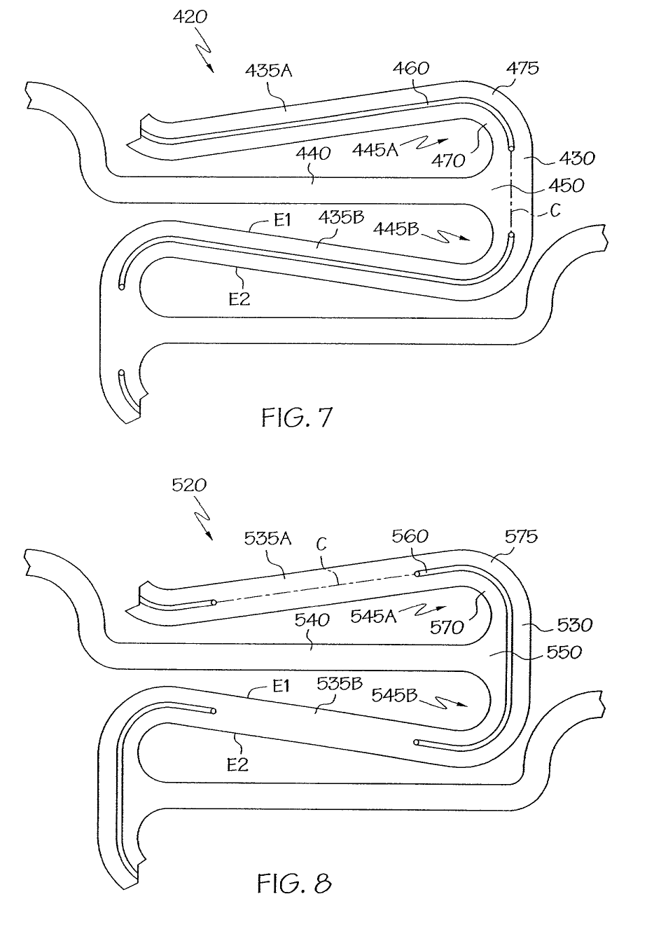

Referring now to FIG. 7, a continuous, longitudinal slot 460 is disposed in the lateral regions 435A and 435B, although in this case the slot is placed along the centerline C such that at all points along its longitude, it is equidistant from the edges E1 and E2. As with the embodiment of FIG. 6, the slot 460 extends partially into the hinge region 430. Simpler manufacturing, promotion of tissue growth, and higher strength within a given strain limit are some of the advantages of this approach, which incorporates the symmetric positioning of the embodiment in FIG. 4 with the continuous, longitudinal features of FIGS. 5 and 6.

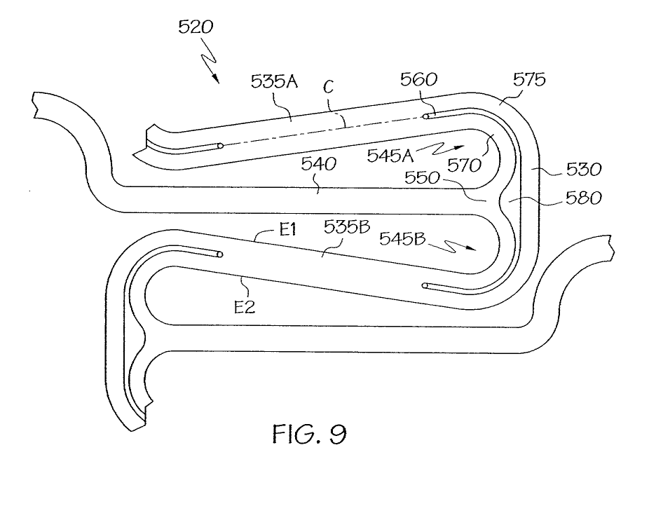

Referring now to FIGS. 8 and 9, a continuous, longitudinal slot 560 is disposed in the lateral regions 535A and 535B. As with the embodiment of FIG. 7, the embodiments of the two present figures include a slot 560 that spans the entire length of one of the regions, in this case, hinge region 530, rather than the lateral regions 435A and 435B of the previous embodiment. Also similar to that of FIG. 7, the slot 560 is placed in an equidistant relationship from the two edges E1 and E2. As with the embodiment shown in FIG. 5, the embodiments of the instant figures provide strain relief throughout the entire hinge region 530, especially in the lateral hinge points 545A, 545B and central hinge point 550. An added feature unique to the embodiment shown in FIG. 9 is the exaggerated slot portion 580, located adjacent the central hinge point 550. Slot portion 580 may have a variable width upon expansion, because it can give way to a different deformation of inner hinge section 570 respective to outer hinge section 530. By this variable width the hinge can be far more flexible compared to a solid one, without taking up too much plastic strain.

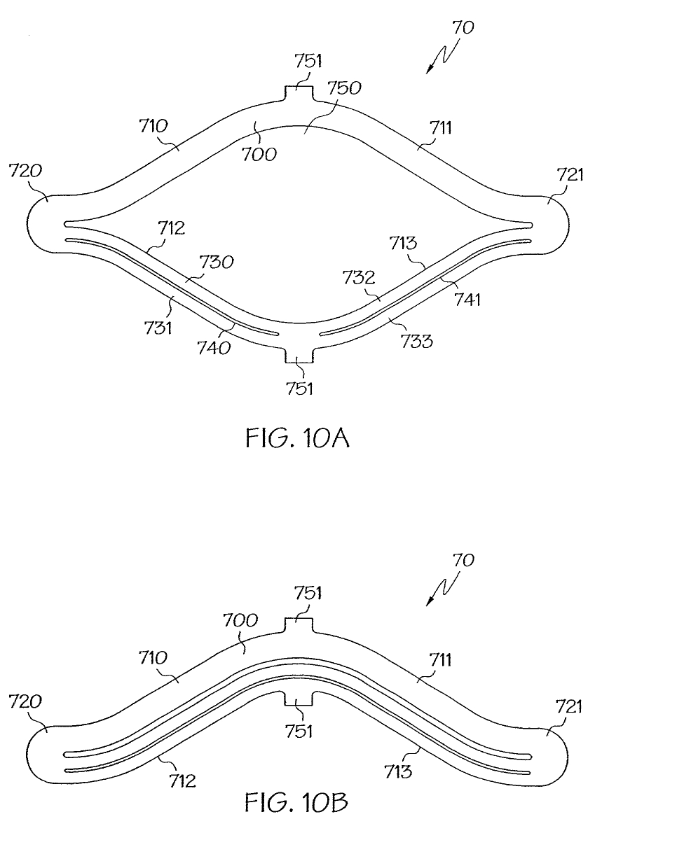

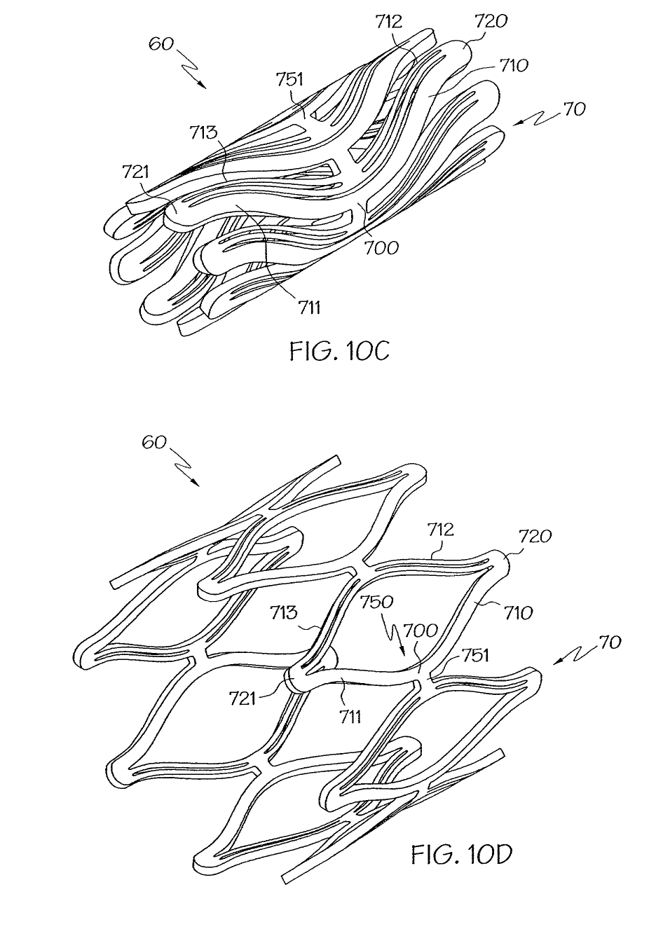

Referring now to FIGS. 10A to 10D, a stent 60 comprises a series of closed unit cells 70, connected at each other to create a closed ring that is expandable by a bistable effect. Methods to create bistable unit cells for a stent have been disclosed in patent application PCT US98/01310. More detail on unit cell 70 can be seen by referring to FIG. 10A, where strut member 700 is made up of two unslotted lateral regions 710 and 711 are shown, with opposing ends of each connected to hinge regions 720 and 721 respectively. The other side includes two slotted lateral regions 712 and 713 with submembers 730 and 731 disposed in the lower left side lateral region 712 and submembers 732 and 733 disposed on the lower right side lateral region 713, divided by slots 740 and 741 respectively. Interconnect regions 751 are used to connect unit cell 70 to adjacent unit cells, as shown in FIGS. 10C and 10D. The special behavior of the unit cell is explained as follows.

The rigidity of the unslotted strut lateral regions 710 and 711 is much higher than for the slotted lateral regions 712 and 713. The effect of splitting lateral regions 712 and 713 in two equal parts of half thickness lowers their rigidity. By deforming the unit cell elastically by compressing interconnect regions 751, 752 toward each other, the upper section with lateral regions 710 and 711 acts as a rigid support for the more flexible lower section slotted lateral regions 712 and 713. During the start of the relative movement between interconnect regions 751, the force will first go up, but after some movement it will go down again, until it becomes zero when the struts are in an intermediate, equilibrium position (not shown) between the positions shown in FIGS. 10A and 10B, after which the unit cell will further collapse automatically until it reaches its end position of FIG. 10B. Around the equilibrium position the unit cell has a negative spring rate, because further compression costs less force. The radial strength of a stent with negative spring rate is maximal at the maximal diameter, which is a typical behavior for stents of this type, and is advantageous in that it forces the deployed stent to occupy the expanded condition, thus minimizing the possibility of collapse during use. Additional advantages of this approach is that the force required to hold such a stent in collapsed state (for example, in a delivery sheath), is minimal, and that friction during delivery from this sheath is minimized.

The unit cell 70, as shown in FIGS. 10A and 10B, is a bistable variant of the embodiments of FIGS. 1 through 9. However, unlike the earlier described embodiments, which rely on plastic deformation around the hinge regions 30, no localized plastic deformation takes place at the various hinge regions 720 and 721 of strut members 700. To achieve this bistable feature, the lateral regions 712 and 713 on only one side of each unit cell has been split in two parts by a pair of longitudinal slots 740 and 741 on both sides of the interconnect region 751 between adjacent unit cells (not shown). In FIG. 10D, a single ring built up from eight bistable unit cells 70 is shown in the expanded state. Such a ring can be very useful in combination with a filter, drug release device, occlusion device, valve or graft material, where the function of the ring is to keep the graft in place in a patient's body. Such a ring can also be combined with more rings in axial direction to build a longer stent. These rings can be of similar repeating patterns or from a different type. Connection is effected via interconnect members or axial connection by means of the graft material itself. It is noted that in the absence of slots, the unit cell would exhibit conventional behavior in that upon the application of a compressive force, each unit cell would be pressed together in a symmetrical way and be flattened out until all struts would be parallel to the main axis of the stent. However, with the addition of slots 740 and 741, compression of the unit cells 70 lead to a configuration as shown in FIGS. 10b and 10C, where the unslotted lateral regions 710 and 711 almost stay undeformed after compression, but the slotted lateral regions 712 and 713 collapse and nest themselves in the concave sections 750 of the unslotted lateral regions 710 and 711. This happens in a special, bistable way if proper unit cell geometry is chosen.

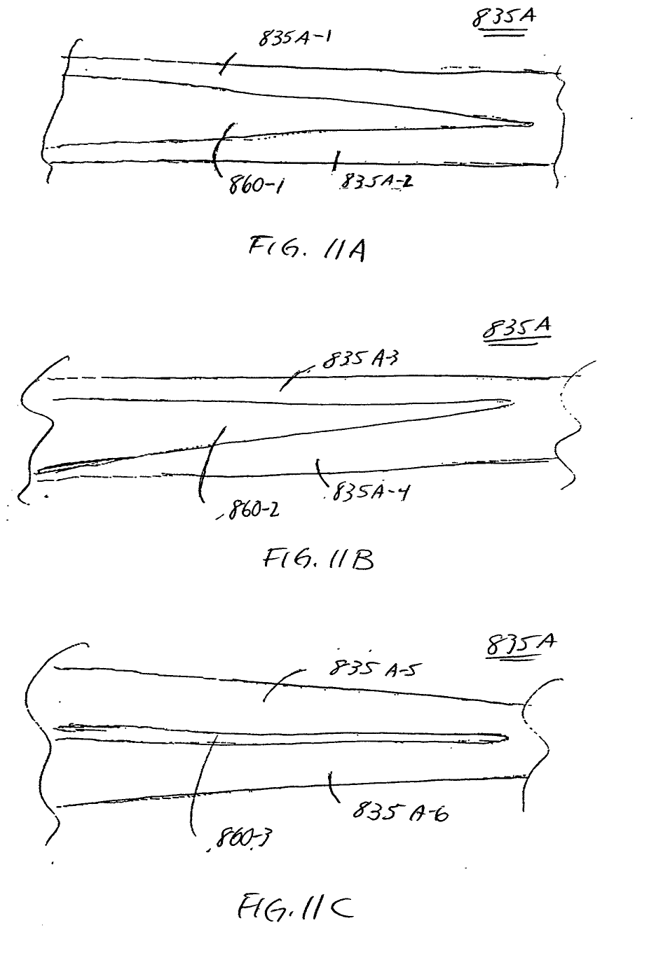

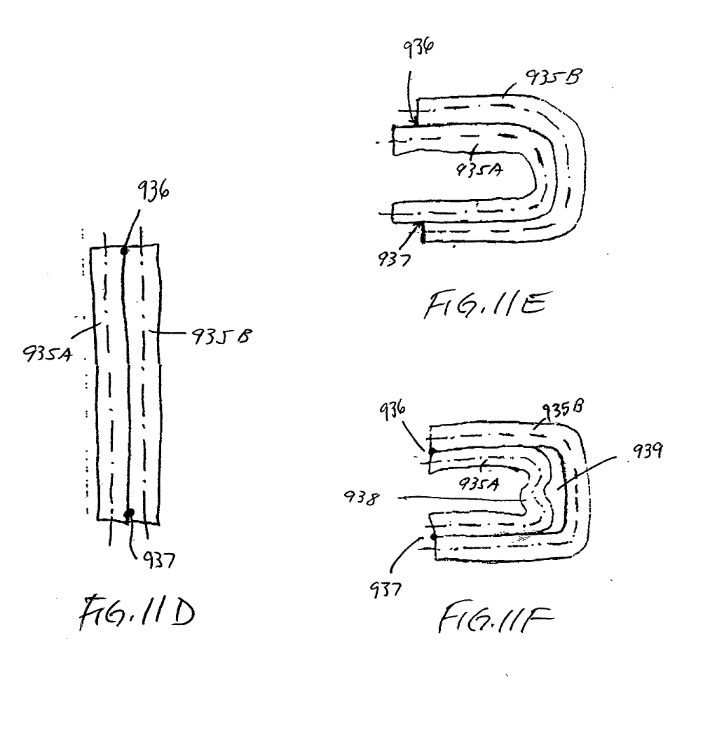

Referring now to FIGS. 11A-11F, variations on the slot and lateral region geometry are shown. As indicated earlier, the stress and strain properties of the various embodiments of the present device can be tailored to meet user needs. Referring specifically to FIGS. 11A-11C, either the lateral regions 835A or 835B (not shown) or the slots 860-1, 860-2 or 860-3 can be either tapered or prismatic. Substruts 835A-1, 835A-2, 835A-3, 835A4, 835A-5 and 835A-6 are defined by the division of lateral region 835A being divided up through at least a portion of its length by slots 860-1, 860-2 or 860-3. Referring specifically to FIGS. 11D, 11E and 11F, two straight struts 935A and 935B have adjacent end points 936 and 937. There are two situations given for bending these two struts together. In FIG. 11E, the end of the struts at the points 936 and 937 are allowed to slide relative to one another because there is no connection between them. Upon bending, each strut will maintain a constant length, measured over its neutral line in the center. The adjacent struts 935A, 935B will nest perfectly in this case and points 936, 937 will move apart. In FIG. 11F, the struts are joined together at end points 936 and 937. Bending of the struts 935A, 935B in this situation will result in a fold 938 in the inner strut, as well as a gap 939 in a shape similar to that of slot 580 shown in FIG. 9, because it has to find a way to store the additional length of the neutral line compared to the situation of FIG. 11E. This additional length equals the sum of the distances between the ends of the adjacent struts 935A and 935B at end points 936 and 937. Upon returning to the straight configuration shown in FIG. 11D, the fold 938 will disappear and gap 939 between the struts 935A and 935B will close again. In the present invention, the gap 939 shape can be adjusted to allow a large shape change upon expansion without excessive plastic deformation. Without the built-in slots according to this invention placed between the adjacent struts (which can act as spring leafs), the shear stress would rapidly increase upon loading, thus preventing the large expansion ratio.

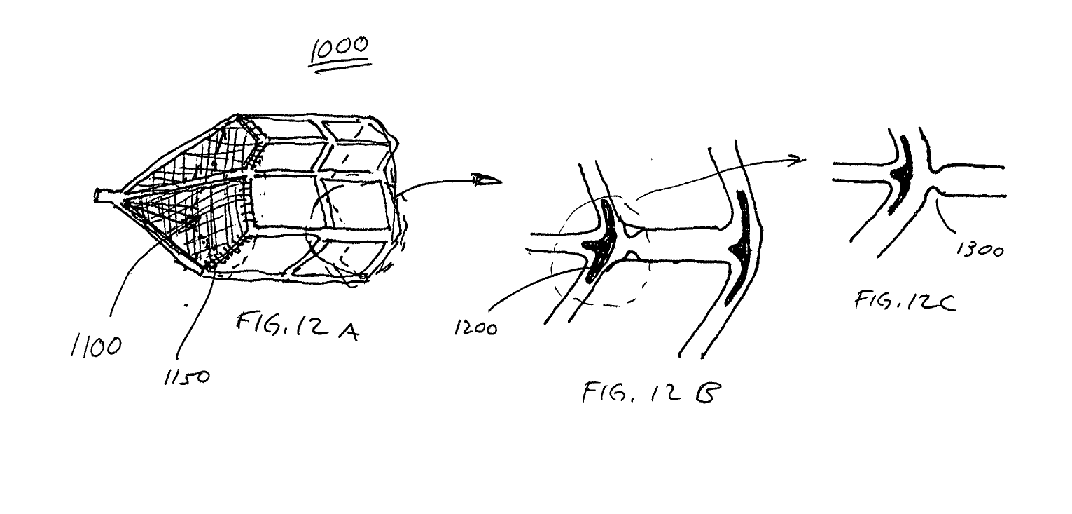

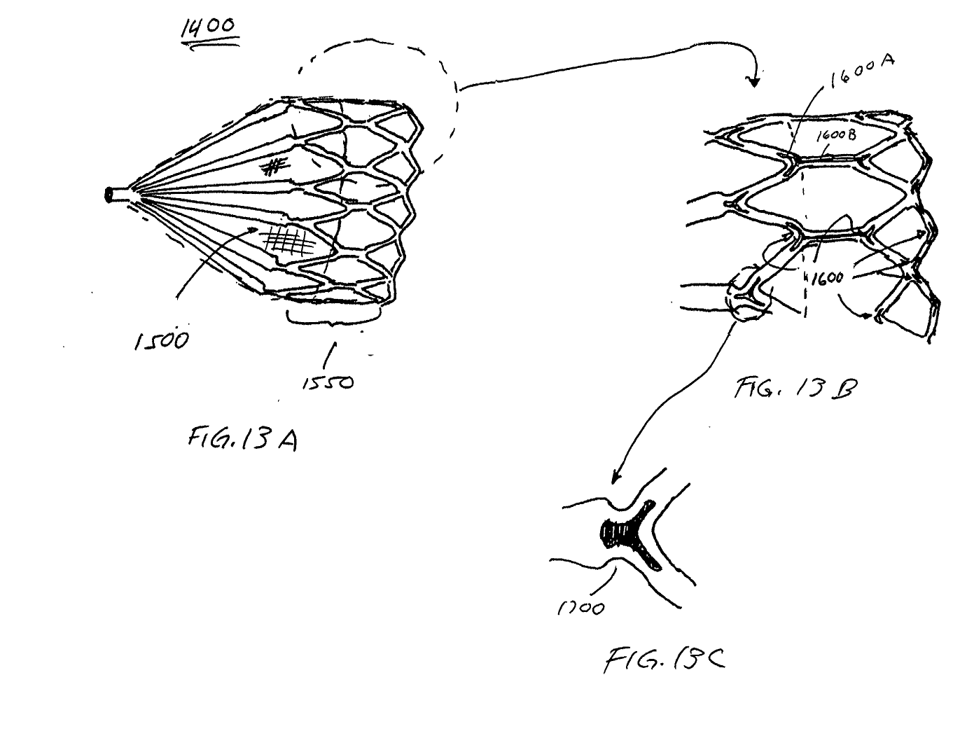

Referring now to FIGS. 12A through 12C and 13A through 13C, adaptations of the expandable housing used to hold various medical inserts is shown. In addition, specific use of gap 939 of FIG. 11F to keep the stress down in the expanded strut members is shown. The second order movements associated with the relative movement between adjacent joined struts can be a significant factor in reducing the possible expansion ratio of the expandable device. Accordingly, properly designed slots can be engineered into the struts to avoid stress buildup when the struts become deformed under expansion. In FIGS. 12A-12C, an expandable device 1000 configured to anchor a valve 1100 is shown. Stitching 1150 is used to connect valve 1100 to expandable device 1000 via apertures (not shown). FIG. 12B shows a representative strut section with an exaggerated slot 1200. The exaggerations serve to effectively lengthen the critical region (in this case the hinge) so that, upon expansion, stress on the struts is kept to a minimum. FIG. 12C shows an optional cutout 1300 that can be used to further reduce interference and related stress buildup. In FIG. 13A, the expandable device 1400 is shown attached to a filter 1500. An example of a ring 1550 is shown in its expanded state, such ring configured to hold the filter 1500 in place. As shown in FIG. 13B, slots 1600 can include exaggerated ends 1600A connected by an elongate central section 1600B. FIG. 13C shows the addition of an optional cutout 1700 to further reduce expansion-related stress buildup.

Having described the invention in detail and by reference to preferred embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

I claim:

1. A unit cell to be used in a stent for inserting into an anatomical lumen and expandable upon insertion into said lumen, said unit cell comprising a plurality of continuous strut members arranged to define a unit cell pattern, wherein said unit cell pattern is arranged in a circumferentially repeating construction defining a radially expandable tubular structure, each of said strut members comprising a plurality of regions, said plurality of regions including:
at least one hinge region; and
a plurality of lateral regions, each of said lateral regions in connection with said hinge region; and
at least one recess disposed in at least one of said plurality of continuous strut members.

2. A unit cell according to claim 1, wherein each of said at least one recess is a slot.

3. A unit cell according to claim 2, wherein said slot is at least one continuous, longitudinal slot.

4. A unit cell according to claim 2, wherein said slot comprises a plurality of discrete slots.

5. A unit cell according to claim 2, wherein the longitudinal axis of each of said slots is positioned asymmetrically with respect to the centerline of said at least one of said plurality of regions.

6. A unit cell according to claim 2, wherein the longitudinal axis of each of said slots is positioned substantially equidistant between opposing edges of said at least one of said plurality of regions.

7. A unit cell according to claim 1, further comprising at least one unit cell interconnect region, said interconnect region including a proximal end in connection with said at least one hinge region, and a distal end.

8. A unit cell according to claim 7 wherein said at least one unit cell interconnect region is substantially elongate.

9. A unit cell according to claim 1, further comprising at least one unit cell interconnect region, said interconnect region including a proximal end in connection with at least one of said plurality of lateral regions, and a distal end.

10. A unit cell according to claim 9, wherein said at least one unit cell interconnect region is substantially elongate.

11. A unit cell according to claim 1, wherein said unit cell is bistable.

12. A ring to be used in a stent for inserting into an anatomical lumen and expandable upon insertion into said lumen, said ring comprising a plurality of continuous strut members arranged to define a unit cell pattern, wherein said unit cell pattern is arranged in a circumferentially repeating construction defining a radially expandable tubular structure configured to expand from a first state into a second state, each of said strut members comprising a plurality of regions, said plurality of regions including:
at least one hinge region;
a plurality of lateral regions, each of said lateral regions in connection with said hinge region; and
at least one recess disposed in at least one of said at least one hinge region or said plurality of lateral regions such that, upon completion of expansion of said unit cell to said predetermined expansion ratio, said tubular structure said first state becomes said tubular structure said second state with a reduced level of strain in said strut members compared to a similar expansion ratio were no said recess present.

13. A ring according to claim 12, wherein said at least one recess is a slot.

14. A ring according to claim 13, further comprising an aperture disposed within at least one of said plurality of regions to facilitate attachment of an insert.

15. A ring according to claim 13, wherein said slot is at least one continuous, longitudinal slot.

16. A ring according to claim 13, wherein said slot comprises a plurality of discrete slots.

17. A ring according to claim 16, wherein each of said plurality of said discrete slots is positioned adjacent at least one lateral hinge point in said hinge region.

18. A ring according to claim 14, wherein the longitudinal axis of each of said slots is positioned asymmetrically with respect to the centerline of said at least one of said plurality of regions.

19. A ring according to claim 14, wherein the longitudinal axis of each of said slots is positioned substantially equidistant between opposing edges of said at least one of said plurality of regions.

20. A ring according to claim 13, further comprising at least one unit cell interconnect region, said interconnect region including a proximal end in connection with said at least one hinge region, and a distal end.

21. A ring according to claim 20, wherein said at least one unit cell interconnect region is substantially elongate.

22. A ring according to claim 17, further comprising at least one unit cell interconnect region, said interconnect region including a proximal end in connection with at least one of said plurality of lateral regions, and a distal end.

23. A ring according to claim 22, wherein said at least one unit cell interconnect region is substantially elongate.

24. A ring according to claim 17, wherein said members are bistable.

25. A ring to be used in a stent for inserting into an anatomical lumen and expandable upon insertion into said lumen, said ring defining an axial dimension and a radial dimension and comprising:
a plurality of continuous strut members arranged to define a unit cell pattern, wherein said unit cell pattern is arranged in a circumferentially repeating construction defining a tubular structure configured to expand in said radial dimension, each of said strut members comprising a plurality of regions, said plurality of regions including:
at least one hinge region;
a plurality of lateral regions angularly offset from said axial dimension, each of said lateral regions in connection with said at least one hinge region;
at least one substantially elongate interconnect region; and
at least one slot disposed in at least one of said plurality of continuous strut members;
a first state of said tubular structure, defining a first diameter; and
a second state of said tubular structure, defining a second diameter, wherein said second diameter is greater than said first diameter by an amount defined by a predetermined expansion ratio, such that, upon completion of expansion of said unit cell to said predetermined expansion ratio, said tubular structure said first state becomes said tubular structure said second state with a reduced level of strain in said strut members compared to a similar expansion ratio were no said recess present.

26. A ring according to claim 25, wherein the longitudinal axis of each of said slots is positioned asymmetrically with respect to the centerline of said at least one of said plurality of regions.

27. A ring according to claim 25, wherein the longitudinal axis of each of said slots is positioned substantially equidistant between opposing edges of said at least one of said plurality of regions.

28. A ring according to claim 25, wherein said at least one slot is at least one continuous, longitudinal slot.

29. A ring according to claim 28, wherein said continuous, longitudinal slot is disposed substantially within said at least one hinge region.

30. A ring according to claim 29, wherein said continuous, longitudinal slot further includes an additional widened portion adjacent a central hinge of said hinge region.

31. A ring according to claim 28, wherein said continuous, longitudinal slot is disposed substantially within said at least one lateral region.

32. A ring according to claim 25, wherein said slots are a plurality of discrete slots.

33. A ring according to claim 32, wherein said plurality of discrete slots are disposed substantially within said at least one hinge region.

34. A ring according to claim 25, wherein said at least one interconnect region includes a proximal end in connection with said at least one hinge region, and a distal end.

35. A ring according to claim 25, wherein said at least one interconnect region includes a proximal end in connection with said plurality of lateral regions, and a distal end.

36. A ring according to claim 25, wherein said members are bistable.

37. A stent for inserting into an anatomical lumen, comprising a plurality of axially repeating unit cells, each unit cell comprising:
a plurality of continuous strut members arranged to define a pattern, wherein said pattern is arranged in a circumferentially repeating construction defining a radially expandable tubular structure, each of said strut members comprising a plurality of regions, said plurality of regions including:
a hinge region;
a plurality of lateral regions, each of said lateral regions in connection with said hinge region; and
at least one recess disposed in at least one of said plurality of continuous strut members;
a first state of said tubular structure, defining a first diameter; and
a second state of said tubular structure, defining a second diameter, wherein said second diameter is greater than said first diameter by an amount defined by a predetermined expansion ratio, such that, upon completion of expansion of said unit cell to said predetermined expansion ratio, said tubular structure said first state becomes said tubular structure said second state with a reduced level of strain in said strut members compared to a similar expansion ratio were no said recess present.

38. A stent according to claim 37, wherein each of said at least one recess is a slot.

39. A stent according to claim 38, wherein a longitudinal axis of each of said slots is positioned substantially equidistant between opposing edges of said at least one of said plurality of regions.

40. A stent according to claim 38, wherein a longitudinal axis of each of said slots is positioned asymmetrically with respect to the centerline of said at least one of said plurality of regions.

41. A stent according to claim 38, wherein said slot is at least one continuous, longitudinal slot.

42. A stent according to claim 41, wherein said continuous, longitudinal slot is disposed substantially within said at least one lateral region.

43. A stent according to claim 41, wherein said continuous, longitudinal slot is disposed substantially within said at least one hinge region.

44. A stent according to claim 43, wherein said continuous, longitudinal slot further includes an additional widened portion adjacent a central hinge of said hinge region.

45. A stent according to claim 38, wherein said slot comprises a plurality of discrete slots.

46. A stent according to claim 39, wherein each of said plurality of said discrete slots is positioned adjacent at least one lateral hinge point in said hinge region.

47. A stent according to claim 37, further comprising at least one substantially elongate unit cell interconnect region, said interconnect region including a proximal end in connection with said hinge region, and a distal end.

48. A stent according to claim 37, further comprising at least one substantially elongate unit cell interconnect region, said interconnect region including a proximal end in connection with said lateral region, and a distal end.

49. A stent according to claim 37, wherein said members are bistable.

50. A stent for inserting into an anatomical lumen and expandable upon insertion into said lumen, said stent comprising:
a plurality of continuous strut members arranged to define a unit cell pattern, wherein said unit cell pattern is arranged in a circumferentially repeating construction defining a radially expandable tubular structure, each of said strut members comprising a plurality of regions, said plurality of regions including:
at least one hinge region;
a plurality of lateral regions angularly offset from said axial dimension, each of said lateral regions in connection with said at least one hinge region;
at least one substantially elongate interconnect region; and
at least one slot disposed in at least one of said plurality of continuous strut members;
a first state of said tubular structure, defining a first diameter; and
a second state of said tubular structure, defining a second diameter, wherein said second diameter is greater than said first diameter by an amount defined by a predetermined expansion ratio, such that, upon completion of expansion of said unit cell to said predetermined expansion ratio, said tubular structure said first state becomes said tubular structure said second state with a reduced level of strain in said strut members compared to a similar expansion ratio were no said recess present.

51. A stent according to claim 50, wherein said at least one interconnect region includes a proximal end in connection with said at least one hinge region, and a distal end.

52. A stent according to claim 50, wherein said at least one interconnect region includes a proximal end in connection with said plurality of lateral regions, and a distal end.

53. A stent according to claim 50, wherein the longitudinal axis of each of said slots is positioned asymmetrically with respect to the centerline of said at least one of said plurality of regions.

54. A stent according to claim 50, wherein the longitudinal axis of each of said slots is positioned substantially equidistant between opposing edges of said at least one of said plurality of regions.

55. A stent according to claim 50, wherein said at least one slot is at least one continuous, longitudinal slot.

56. A stent according to claim 55, wherein said continuous, longitudinal slot is disposed substantially within said lateral region.

57. A stent according to claim 55, wherein said continuous, longitudinal slot is disposed substantially within said hinge region.

58. A stent according to claim 55, wherein said continuous, longitudinal slot further includes an additional widened portion adjacent a central hinge of said hinge region.

59. A stent according to claim 50, wherein said at least one slot comprises a plurality of discrete slots.

60. A stent according to claim 50, wherein said members are bistable.

61. An expandable medical device configured for use as a housing for intraluminal inserts, said expandable medical device including a first state defining a first diameter and a second state defining a second diameter, wherein said second diameter is greater than said first diameter by an amount defined by a predetermined expansion ratio, said expandable medical device comprising:
a plurality of continuous strut members arranged to define a generally repeating pattern, wherein said generally repeating pattern is arranged such that each of said continuous strut members comprise a plurality of regions, said plurality of regions including:
a hinge region;
a plurality of lateral regions, each of said lateral regions in connection with said hinge region;
an interconnect region to connect said generally repeating pattern with an adjacent repeating pattern; and
at least one slot disposed in at least said hinge region or said plurality of lateral regions.

62. An expandable medical device according to claim 61, further comprising at least one aperture disposed in at least one of said plurality of regions to facilitate the attachment of said expandable medical device to said insert.

63. An expandable medical device according to claim 62, wherein said insert is selected from the group consisting of graft material, drug release device, valves, occlusion device and filters.

64. An expandable medical device according to claim 62, wherein said attachment to said insert material is configured to be accomplished through the use of materials from the group consisting of stitches, sewing wire and rivets.

65. A method of expanding an expandable housing, comprising:
configuring a length of generally tubular, expandable housing defined by a substantially lengthwise axial dimension and a radial dimension, said expandable housing configured to expand from a first state of said tubular structure defining a first diameter to a second state of said tubular structure defining a second diameter such that said second diameter is greater than said first diameter by an amount defined by a predetermined expansion ratio, said expandable housing possessive of a repeating unit cell, each of which comprises a plurality of continuous strut members, each defining a plurality of regions, said plurality of regions including:
a hinge region;
a plurality of lateral regions, each of said lateral regions in connection with said hinge region; and
at least one slot disposed in at least one of said plurality of continuous strut members;
placing said expandable housing in a predetermined location; and
expanding said expandable housing such that said housing changes from said first state into said second state.

66. A method according to claim 65, wherein prior to said step of placing said expandable housing in a predetermined location, said method further comprises the additional steps of:
inserting a catheter into an inner wall of said expandable housing; and
providing a conduit for the introduction of an expansion fluid into said catheter, said conduit in fluid communication with an external fluid pressure supply.

67. A method according to claim 66, wherein after said step of expanding said expandable housing such that said housing changes from said first state into said second state, said method further comprises the additional steps of:
deflating said catheter; and
withdrawing said deflated catheter from said expandable housing.

68. A method according to claim 65, wherein said expandable housing is self-expanding from said first state into said second state.

69. A method according to claim 68, wherein sometime prior to said step of expanding said expandable housing such that said housing changes from said first state into said second state, said method further comprises the additional step of restraining said expandable housing.

70. A method according to claim 68, wherein sometime prior to said step of expanding said expandable housing such that said housing changes from said first state into said second state, said method further comprises the additional step of separating said expandable housing from said restraint.

71. A method according to claim 65, wherein said slots are defined by a series of discrete slots positioned substantially equidistant between opposing edges of said strut members.

72. A method according to claim 65, wherein said slots are defined by a series of discrete slots positioned asymmetrically with respect to the centerline of said strut members.

73. A method according to claim 65, wherein said slots are defined by longitudinal and continuous slots throughout an entire length of at least one of a hinge region, a lateral region, or an interconnect region of each of said strut members, said slots positioned substantially equidistant between opposing edges of said strut members.

74. A method according to claim 65, wherein said slots are longitudinal and continuous throughout an entire length of at least one of a hinge region, a lateral region, or an interconnect region of each of said strut members, said slots positioned asymmetrically with respect to the centerline of said strut members.

75. A method according to claim 65, further comprising the steps of:
placing at least one aperture in at least one of said plurality of regions; and
attaching an insert to said expandable housing via said at least one aperture.

76. A method according to claim 75, wherein said attaching is accomplished through the use of materials from the group consisting of stitches, sewing wire or rivets.

77. A method according to claim 76, wherein said insert is selected from the group consisting of graft material, occlusion device, drug release device, valves and filters.

78. A method according to claim 65, wherein said lateral members are angularly offset relative to said axial dimension of said expandable housing

79. A method according to claim 65, wherein at least some of said slots or some of said lateral regions are non-prismatic in shape.

80. A bistable unit cell to be used in a stent for inserting into an anatomical lumen and expandable upon insertion into said lumen, said unit cell comprising:
a solid elongate strut member;
a slotted elongate strut member capable of a first bistable state and a second bistable state, wherein said slotted elongate strut member is nested with said solid elongate strut member in said first bistable state, and expanded away from said solid elongate strut member in said second bistable state;
a plurality of axially spaced hinge members, each defined by joined adjacent ends of said solid elongate strut member and said slotted elongate strut member, said plurality of axially spaced hinge members defining an elongate aperture configured as a curvilinear slot in said first bistable state and as an expanded opening in said second bistable state.

81. A bistable ring to be used in a stent for inserting into an anatomical lumen and expandable upon insertion into said lumen, said bistable ring comprising:
a plurality of circumferentially repeating unit cells, each of said unit cells comprising:
a solid elongate strut member;
a slotted elongate strut member capable of a first bistable state and a second bistable state, wherein said slotted elongate strut member is nested with said solid elongate strut member in said first bistable state, and expanded away from said solid elongate strut member in said second bistable state; and
a plurality of axially spaced hinge members, each defined by joined adjacent ends of said solid elongate strut member and said slotted elongate strut member, said plurality of axially spaced hinge members defining an elongate aperture configured as a curvilinear slot in said first bistable state and as an expanded opening in said second bistable state; and
an interconnect region disposed between adjacent bistable unit cells such that said adjacent bistable unit cells are configured to cooperatively expand when said bistable ring expands from said first bistable state into said second bistable state.

82. A bistable stent for inserting into an anatomical lumen and expandable upon insertion into said lumen, said bistable stent comprising:
a plurality of axially aligned bistable rings, each of said bistable rings formed by a plurality of interconnected unit cells arranged in a circumferentially repeating pattern, each of said unit cells comprising:
a solid elongate strut member;
a slotted elongate strut member capable of a first bistable state and a second bistable state, wherein said slotted elongate strut member is nested with said solid elongate strut member in said first bistable state, and expanded away from said solid elongate strut member in said second bistable state; and
a plurality of axially spaced hinge members, each defined by joined adjacent ends of said solid elongate strut member and said slotted elongate strut member, said plurality of axially spaced hinge members defining an elongate aperture configured as a curvilinear slot in said first bistable state and as an expanded opening in said second bistable state;
a first interconnect region disposed between adjacent bistable unit cells such that said adjacent bistable unit cells are configured to cooperatively expand when said bistable ring expands from said first bistable state into said second bistable state; and
a second interconnect region disposed between axially adjacent hinge members such that said plurality of axially aligned bistable rings are connected together to form said bistable stent.
